(19) United States
(12) Patent Application Publication
(10) Pub. No.: US 2012/0041518 A1
(43) Pub. Date: Feb. 16, 2012

(54) NEUROSTIMULATION SYSTEM AND METHOD WITH GRAPHICALLY MANIPULATABLE STIMULATION TARGET

(75) Inventors: Chester Kim, Lynnwood, WA (US); Dongchul Lee, Valencia, CA (US)

(73) Assignee: BOSTON SCIENTIFIC NEUROMODULATION CORPORATION, Valencia, CA (US)

(21) Appl. No.: 13/197,987

(22) Filed: Aug. 4, 2011

Related U.S. Application Data
(60) Provisional application No. 61/373,756, filed on Aug. 13, 2010.

(51) Int. Cl. A61N 1/08

(52) U.S. Cl. 607/60

(57) ABSTRACT

An external control device for use with a neurostimulation device having a plurality of electrodes carried by at least one neurostimulation lead. The external control device comprises a display screen configured for displaying at least one object relative to a graphical representation of the neurostimulation lead(s). The object(s) represents an abstraction of a stimulation target. The external control device further comprises detection circuitry configured for detecting an actuation event that includes placing at least one pointing element in proximity to the object(s). The external control device further comprises processing circuitry configured for manipulating the object(s) on the display screen in response to the detection of the actuation event, thereby modifying the stimulation target abstraction, and for generating a set of stimulation parameters that emulates the modified stimulation target abstraction. The external control device further comprises output circuitry configured for transmitting the set of stimulation parameters to the neurostimulation device.

### RELATED APPLICATION DATA

The present application claims the benefit under 35 U.S.C. §119 to U.S. provisional patent application Ser. No. 61/373,756, filed Aug. 13, 2010. The foregoing application is hereby incorporated by reference into the present application in its entirety.

### FIELD OF THE INVENTION

The present inventions relate to tissue stimulation systems, and more particularly, to neurostimulation systems for programming neurostimulation leads.

### BACKGROUND OF THE INVENTION

Implantable neurostimulation systems have proven therapeutic in a wide variety of diseases and disorders. Pacemakers and Implantable Cardiac Defibrillators (ICDs) have proven highly effective in the treatment of a number of cardiac conditions (e.g., arrhythmias). Spinal Cord Stimulation (SCS) systems have long been accepted as a therapeutic modality for the treatment of chronic pain syndromes, and the application of tissue stimulation has begun to expand to additional applications such as angina pectoralis and incontinence. Deep Brain Stimulation (DBS) has also been applied therapeutically for well over a decade for the treatment of refractory chronic pain syndromes, and DBS has also recently been applied in additional areas such as movement disorders and epilepsy. Further, in recent investigations, Peripheral Nerve Stimulation (PNS) systems have demonstrated efficacy in the treatment of chronic pain syndromes and incontinence, and a number of additional applications are currently under investigation. Furthermore, Functional Electrical Stimulation (FES) systems, such as the Freehand system by NeuroControl (Cleveland, Ohio), have been applied to restore some functionality to paralyzed extremities in spinal cord injury patients.

These implantable neurostimulation systems typically include one or more electrode carrying stimulation leads, which are implanted at the desired stimulation site, and a neurostimulator (e.g., an implantable pulse generator (IPG)) implanted remotely from the stimulation site, but coupled either directly to the stimulation lead(s) or indirectly to the stimulation lead(s) via a lead extension. The neurostimulation system may further comprise an external control device to remotely instruct the neurostimulator to generate electrical stimulation pulses in accordance with selected stimulation parameters.

Electrical stimulation energy may be delivered from the neurostimulator to the electrodes in the form of an electrical pulsed waveform. Thus, stimulation energy may be controllably delivered to the electrodes to stimulate neural tissue. The combination of electrodes used to deliver electrical pulses to the targeted tissue constitutes an electrode combination, with the electrodes capable of being selectively programmed to act as anodes (positive), cathodes (negative), or left off (zero). In other words, an electrode combination represents the polarity being positive, negative, or zero. Other parameters that may be controlled or varied include the amplitude, width, and rate of the electrical pulses provided through the electrode array. Each electrode combination, along with the electrical pulse parameters, can be referred to as a “stimulation parameter set.”

With some neurostimulation systems, and in particular, those with independently controlled current or voltage sources, the distribution of the current to the electrodes (including the case of the neurostimulator, which may act as an electrode) may be varied such that the current is supplied via numerous different electrode configurations. In different configurations, the electrodes may provide current or voltage in different relative percentages of positive and negative current or voltage to create different electrical current distributions (i.e., fractionalized electrode combinations).

As briefly discussed above, an external control device can be used to instruct the neurostimulator to generate electrical stimulation pulses in accordance with the selected stimulation parameters. Typically, the stimulation parameters programmed into the neurostimulator can be adjusted by manipulating controls on the external control device to modify the electrical stimulation provided by the neurostimulator system to the patient. Thus, in accordance with the stimulation parameters programmed by the external control device, electrical pulses can be delivered from the neurostimulator to the stimulation electrode(s) to stimulate or activate a volume of tissue in accordance with a set of stimulation parameters and provide the desired efficacious therapy to the patient. The best stimulus parameter set will typically be one that delivers stimulation energy to the volume of tissue that must be stimulated in order to provide the therapeutic benefit (e.g., treatment of pain), while minimizing the volume of non-target tissue that is stimulated.

However, the number of electrodes available, combined with the ability to generate a variety of complex stimulation pulses, presents a huge selection of stimulation parameter sets to the clinician or patient. For example, if the neurostimulation system to be programmed has an array of sixteen electrodes, millions of stimulation parameter sets may be available for programming into the neurostimulation system. Today, neurostimulation system may have up to thirty-two electrodes, thereby exponentially increasing the number of stimulation parameters sets available for programming.

To facilitate such selection, the clinician generally programs the neurostimulator through a computerized programming system. This programming system can be a self-contained hardware/software system, or can be defined predominantly by software running on a standard personal computer (PC). The PC or custom hardware may actively control the characteristics of the electrical stimulation generated by the neurostimulator to allow the optimum stimulation parameters to be determined based on patient feedback or other means and to subsequently program the neurostimulator with the optimum stimulation parameter set or sets, which will typically be those that stimulate all of the target tissue in order to provide the therapeutic benefit, yet minimizes the volume of non-target tissue that is stimulated. The computerized programming system may be operated by a clinician attending the patient in several scenarios.

For example, in order to achieve an effective result from SCS, the lead or leads must be placed in a location, such that the electrical stimulation will cause paresthesia. The paresthesia induced by the stimulation and perceived by the patient should be located in approximately the same place in the patient's body as the pain that is the target of treatment. If a lead is not correctly positioned, it is possible that the patient will receive little or no benefit from an implanted SCS system. Thus, correct lead placement can mean the difference between effective and ineffective pain therapy. When electrical leads are implanted within the patient, the computerized programming system, in the context of an operating room (OR) mapping procedure, may be used to instruct the neurostimulator to apply electrical stimulation to test placement of the leads and/or electrodes, thereby assuring that the leads and/or electrodes are implanted in effective locations within the patient.

Once the leads are correctly positioned, a fitting procedure, which may be referred to as a navigation session, may be performed using the computerized programming system to program the external control device, and if applicable the neurostimulator, with a set of stimulation parameters that best addresses the painful site. Thus, the navigation session may be used to pinpoint the stimulation region or areas correlating to the pain. Such programming ability is particularly advantageous for targeting the tissue during implantation, or after implantation should the leads gradually or unexpectedly move that would otherwise relocate the stimulation energy away from the target site. By reprogramming the neurostimulator (typically by independently varying the stimulation energy on the electrodes), the stimulation region can often be moved back to the effective pain site without having to re-operate on the patient in order to reposition the lead and its electrode array. When adjusting the stimulation region relative to the tissue, it is desirable to make small changes in the proportions of current, so that changes in the spatial recruitment of nerve fibers will be perceived by the patient as being smooth and continuous and to have incremental targeting capability.

One known computerized programming system for SCS is called the Bionic Navigator®, available from Boston Scientific Neuromodulation Corporation. The Bionic Navigator® is a software package that operates on a suitable PC and allows clinicians to program stimulation parameters into an external handheld programmer (referred to as a remote control). Each set of stimulation parameters, including fractionalized current distribution to the electrodes (as percentage cathodic current, percentage anodic current, or off), may be stored in both the Bionic Navigator® and the remote control and combined into a stimulation program that can then be used to stimulate multiple regions within the patient.

Prior to creating the stimulation programs, the Bionic Navigator® may be operated by a clinician in a “manual mode” to manually select the percentage cathodic current and percentage anodic current flowing through the electrodes, or may be operated by the clinician in an “automated mode” to electrically “steer” the current along the implanted leads in real-time (e.g., using a joystick or joystick-like controls), thereby allowing the clinician to determine the most efficacious stimulation parameter sets that can then be stored and eventually combined into stimulation programs. The Bionic Navigator® may use one of two ways to electrically steer the current along the implanted leads.

In one method, known as “weaving,” the anode or anodes are moved around the cathode, while the cathode slowly progresses down the sequence of electrodes. In another method, known as “panning,” a pre-defined electrode combination is shifted down the sequence of electrodes without changing the basic form of the electrode combination. Since these current steering methods may have different clinical uses (e.g., finding the “sweet spot” in the case of panning, or shaping the electrical field around the cathode in the case of weaving), the user may have to switch between these two methods, resulting in electrode combinations that have not been tested, and thus, therapeutic options not explored and possibly suboptimal outcomes.

In one novel method, described in U.S. Provisional Application Ser. No. 61/257,753, entitled “System and Method for Mapping Arbitrary Electric Fields to Pre-existing Lead Electrodes,” which is incorporated herein by reference, a stimulation target in the form of an ideal bipole or tripole is defined and the stimulation parameters, including the fractionalized current values on each of the electrodes, is computationally determined in a manner that emulates these ideal poles. It can be appreciated that current steering can be implemented by moving the ideal bipole or tripole about the leads, such that the appropriate fractionalized current values for the electrodes are computed for each of the various positions of the ideal bipole or tripole. Different types of current steering, including weaving and panning, can be implemented in this technique.

While the computation of stimulation parameters to emulate ideal bipoles and tripoles is quite useful, there remains a need for an intuitive control mechanism that allows a user to easily move a stimulation target amongst electrodes, while also providing an efficient manner for switching between different current steering methods, in which different current steering.

### SUMMARY OF THE INVENTION

In accordance with the present inventions, an external control device for use with a neurostimulation device having a plurality of electrodes carried by at least one neurostimulation lead is provided.

The external control device comprises a display screen configured for displaying at least one object relative to a graphical representation of the at least one neurostimulation lead. The object(s) represent an abstraction of a stimulation target (e.g., an ideal multi-pole, an ideal electrical field, an ideal stimulation region, etc.).

The external control device further comprises detection circuitry configured for detecting an actuation event that includes placing at least one pointing element in proximity to the object(s). The actuation event may further include moving the pointing element(s) across the display screen. In one embodiment, each of the pointing element(s) is a virtual pointing element, in which case, the actuation event may include graphically placing the virtual pointing element(s) in proximity to the object(s). In another embodiment, each of the pointing element(s) is an actual pointing element, in which case, the actuation event may include physically placing the actual pointing element(s) in proximity to the object(s).

The external control device further comprises processing circuitry configured for manipulating the object(s) on the display screen in response to the detection of the actuation event, thereby modifying the stimulation target abstraction, and for generating a set of stimulation parameters that emulates the modified stimulation target abstraction. The external control device further comprises output circuitry (e.g., telemetry circuitry) configured for transmitting the set of stimulation parameters to the neurostimulation device. The external control device may have a housing containing the display screen, detection circuitry, processing circuitry, and output circuitry.

In one embodiment, the processing circuitry is configured for manipulating the object(s) on the display screen by displacing the object(s) on the display screen. If there is a plurality of objects, the processing circuitry may be configured for displacing the objects relative to each other. In another embodiment, the processing circuitry is configured for manipulating the object(s) on the display screen by modifying the geometry of the at least one object; for example, by expanding or contract the object(s). In an optional embodiment, the external control device may comprise another control element configured for being actuated, in which case, the processing circuitry may be configured for generating the stimulation parameter set in accordance with a first current steering algorithm in response to the detection of the actuation event, and for generating another stimulation parameter set in accordance with a second current steering algorithm in response to the actuation of the other control element.

In one embodiment, the display screen is configured for displaying a graphical control element adjacent each of the object(s), in which case, the actuation event may include placing the pointing element(s) respectively on the control element(s) and clicking or tapping the control element(s), and the processing circuitry may be configured for displacing the control element(s) in unison with the manipulation of the object(s), such that the graphical control element remains displayed adjacent the manipulated object(s).

If the pointing element(s) are a plurality of actual pointing elements, the actuating event may include placing the actual pointing elements concurrently in proximity to the object(s) and moving the actual pointing elements relative to each other across the display screen. If there is plurality of objects, the actuating event may include placing the actual pointing elements concurrently in proximity to the respective objects, in which case, the processing circuitry may be configured for manipulating the objects on the display screen by moving the objects relative to each other. If there is a single object, the actuating event may include placing the actual pointing elements concurrently in proximity to the single object, in which case, the processing circuitry may be configured for manipulating the object on the display screen by modifying the geometry of the object.

Other and further aspects and features of the invention will be evident from reading the following detailed description of the preferred embodiments, which are intended to illustrate, not limit, the invention.

### BRIEF DESCRIPTION OF THE DRAWINGS

The drawings illustrate the design and utility of preferred embodiments of the present invention, in which similar elements are referred to by common reference numerals. In order to better appreciate how the above-recited and other advantages and objects of the present inventions are obtained, a more particular description of the present inventions briefly described above will be rendered by reference to specific embodiments thereof, which are illustrated in the accompanying drawings. Understanding that these drawings depict only typical embodiments of the invention and are not therefore to be considered limiting of its scope, the invention will be described and explained with additional specificity and detail through the use of the accompanying drawings in which:

FIG. 1 is a plan view of a Spinal cord Stimulation (SCS) system constructed in accordance with one embodiment of the present inventions;

FIG. 2 is a perspective view of the arrangement of the SCS system of FIG. 1 with respect to a patient;

FIG. 3 is a profile view of an implantable pulse generator (IPG) and percutaneous leads used in the SCS system of FIG. 1;

FIG. 4 is front view of a remote control (RC) used in the SCS system of FIG. 1;

FIG. 5 is a block diagram of the internal components of the RC of FIG. 4;

FIG. 6 is a block diagram of the internal components of a clinician's programmer (CP) used in the SCS system of FIG. 1;

FIG. 7 is a plan view of a user interface of the CP of FIG. 6 for programming the IPG of FIG. 3;

FIGS. 8A-8J are plan views respectively illustrating the manipulation of ideal poles in a programming screen generated by the user interface of FIG. 7;

FIGS. 9A-9F are plan views respectively illustrating the manipulation of an ideal electrical field in the programming screen generated by the user interface of FIG. 7; and

FIGS. 10A-10B are plan views respectively illustrating the manipulation of an ideal stimulation region in the programming screen generated by the user interface of FIG. 7.

### DETAILED DESCRIPTION OF THE EMBODIMENTS

The description that follows relates to a spinal cord stimulation (SCS) system. However, it is to be understood that while the invention lends itself well to applications in SCS, the invention, in its broadest aspects, may not be so limited. Rather, the invention may be used with any type of implantable electrical circuitry used to stimulate tissue. For example, the present invention may be used as part of a pacemaker, a defibrillator, a cochlear stimulator, a retinal stimulator, a stimulator configured to produce coordinated limb movement, a cortical stimulator, a deep brain stimulator, peripheral nerve stimulator, microstimulator, or in any other neurostimulator configured to treat urinary incontinence, sleep apnea, shoulder sublaxation, headache, etc.

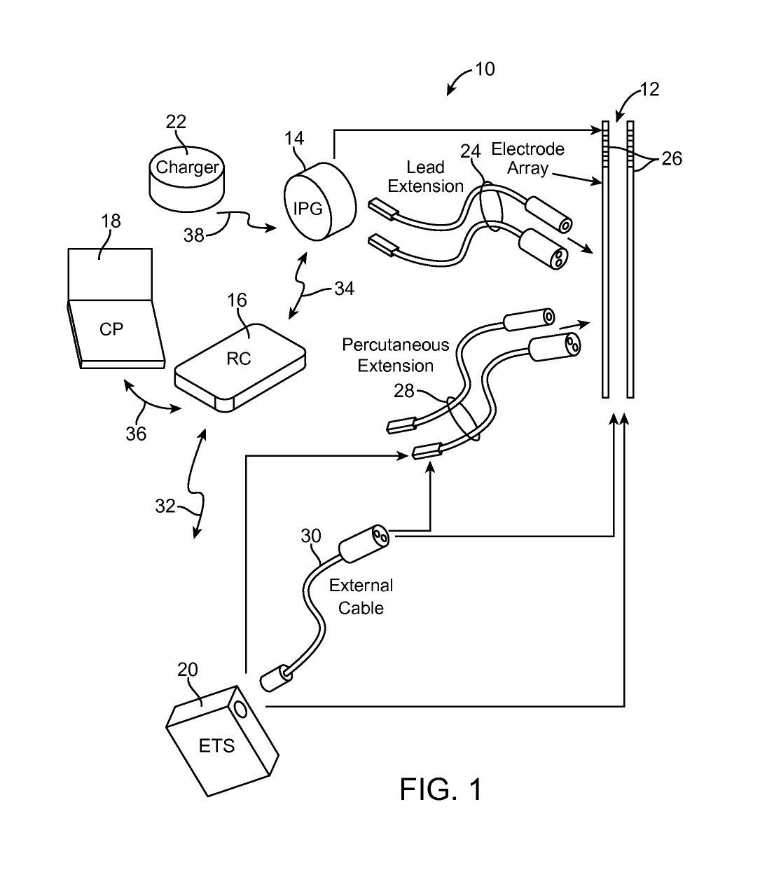

Turning first to FIG. 1, an exemplary SCS system 10 generally includes a plurality (in this case, two) of implantable neurostimulation leads 12, an implantable pulse generator (IPG) 14, an external remote controller RC 16, a clinician's programmer (CP) 18, an external trial stimulator (ETS) 20, and an external charger 22.

The IPG 14 is physically connected via one or more percutaneous lead extensions 24 to the neurostimulation leads 12, which carry a plurality of electrodes 26 arranged in an array. In the illustrated embodiment, the neurostimulation leads 12 are percutaneous leads, and to this end, the electrodes 26 are arranged in-line along the neurostimulation leads 12. As will be described in further detail below, the IPG 14 includes pulse generation circuitry that delivers electrical stimulation energy in the form of a pulsed electrical waveform (i.e., a temporal series of electrical pulses) to the electrode array 26 in accordance with a set of stimulation parameters.

The ETS 20 may also be physically connected via the percutaneous lead extensions 28 and external cable 30 to the neurostimulation leads 12. The ETS 20, which has similar pulse generation circuitry as the IPG 14, also delivers electrical stimulation energy in the form of a pulse electrical waveform to the electrode array 26 accordance with a set of stimulation parameters. The major difference between the ETS 20 and the IPG 14 is that the ETS 20 is a non-implantable device that is used on a trial basis after the neurostimulation leads 12 have been implanted and prior to implantation of the IPG 14, to test the responsiveness of the stimulation that is to be provided. Further details of an exemplary ETS are described in U.S. Pat. No. 6,895,280, which is expressly incorporated herein by reference.

The RC 16 may be used to telemetrically control the ETS 20 via a bi-directional RF communications link 32. Once the IPG 14 and neurostimulation leads 12 are implanted, the RC 16 may be used to telemetrically control the IPG 14 via a bi-directional RF communications link 34. Such control allows the IPG 14 to be turned on or off and to be programmed with different stimulation parameter sets. The IPG 14 may also be operated to modify the programmed stimulation parameters to actively control the characteristics of the electrical stimulation energy output by the IPG 14. As will be described in further detail below, the CP 18 provides clinician detailed stimulation parameters for programming the IPG 14 and ETS 20 in the operating room and in follow-up sessions.

The CP 18 may perform this function by indirectly communicating with the IPG 14 or ETS 20, through the RC 16, via an IR communications link 36. Alternatively, the CP 18 may directly communicate with the IPG 14 or ETS 20 via an RF communications link (not shown). The clinician detailed stimulation parameters provided by the CP 18 are also used to program the RC 16, so that the stimulation parameters can be subsequently modified by operation of the RC 16 in a stand-alone mode (i.e., without the assistance of the CP 18).

The external charger 22 is a portable device used to transcutaneously charge the IPG 14 via an inductive link 38. For purposes of brevity, the details of the external charger 22 will not be described herein. Details of exemplary embodiments of external chargers are disclosed in U.S. Pat. No. 6,895,280, which has been previously incorporated herein by reference. Once the IPG 14 has been programmed, and its power source has been charged by the external charger 22 or otherwise replenished, the IPG 14 may function as programmed without the RC 16 or CP 18 being present.

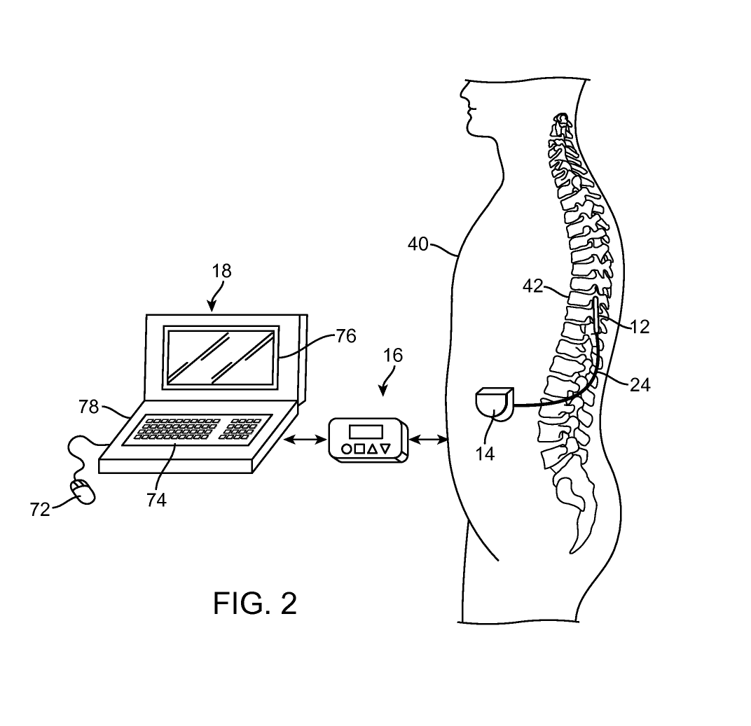

As shown in FIG. 2, the electrode leads 12 are implanted within the spinal column 42 of a patient 40. The preferred placement of the electrode leads 12 is adjacent, i.e., resting upon, the spinal cord area to be stimulated. Due to the lack of space near the location where the electrode leads 12 exit the spinal column 42, the IPG 14 is generally implanted in a surgically-made pocket either in the abdomen or above the buttocks. The IPG 14 may, of course, also be implanted in other locations of the patient's body. The lead extensions 24 facilitate locating the IPG 14 away from the exit point of the electrode leads 12. As there shown, the CP 18 communicates with the IPG 14 via the RC 16.

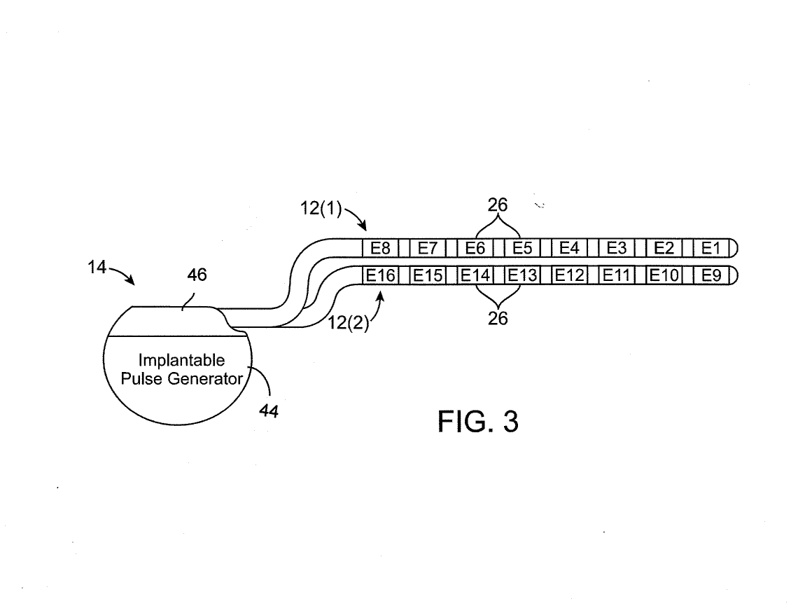

Referring now to FIG. 3, the external features of the neurostimulation leads 12 and the IPG 14 will be briefly described. One of the neurostimulation leads 12(1) has eight electrodes 26 (labeled E1-E8), and the other neurostimulation lead 12(2) has eight electrodes 26 (labeled E9-E16). The actual number and shape of leads and electrodes will, of course, vary according to the intended application. The IPG 14 comprises an outer case 44 for housing the electronic and other components (described in further detail below), and a connector 46 to which the proximal ends of the neurostimulation leads 12 mates in a manner that electrically couples the electrodes 26 to the electronics within the outer case 44. The outer case 44 is composed of an electrically conductive, biocompatible material, such as titanium, and forms a hermetically sealed compartment wherein the internal electronics are protected from the body tissue and fluids. In some cases, the outer case 44 may serve as an electrode.

The IPG 14 includes a battery and pulse generation circuitry that delivers the electrical stimulation energy in the form of a pulsed electrical waveform to the electrode array 26 in accordance with a set of stimulation parameters programmed into the IPG 14. Such stimulation parameters may comprise electrode combinations, which define the electrodes that are activated as anodes (positive), cathodes (negative), and turned off (zero), percentage of stimulation energy assigned to each electrode (fractionalized electrode combinations), and electrical pulse parameters, which define the pulse amplitude (measured in milliamps or volts depending on whether the IPG 14 supplies constant current or constant voltage to the electrode array 26), pulse width (measured in microseconds), and pulse rate (measured in pulses per second).

Electrical stimulation will occur between two (or more) activated electrodes, one of which may be the IPG case. Simulation energy may be transmitted to the tissue in a monopolar or multipolar (e.g., bipolar, tripolar, etc.) fashion. Monopolar stimulation occurs when a selected one of the lead electrodes 26 is activated along with the case 44 of the IPG 14, so that stimulation energy is transmitted between the selected electrode 26 and case. Bipolar stimulation occurs when two of the lead electrodes 26 are activated as anode and cathode, so that stimulation energy is transmitted between the selected electrodes 26. For example, electrode E3 on the first lead 12 may be activated as an anode at the same time that electrode E11 on the second lead 12 is activated as a cathode. Tripolar stimulation occurs when three of the lead electrodes 26 are activated, two as anodes and the remaining one as a cathode, or two as cathodes and the remaining one as an anode. For example, electrodes E4 and E5 on the first lead 12 may be activated as anodes at the same time that electrode E12 on the second lead 12 is activated as a cathode.

In the illustrated embodiment, IPG 14 can individually control the magnitude of electrical current flowing through each of the electrodes. In this case, it is preferred to have a current generator, wherein individual current-regulated amplitudes from independent current sources for each electrode may be selectively generated. Although this system is optimal to take advantage of the invention, other stimulators that may be used with the invention include stimulators having voltage regulated outputs. While individually programmable electrode amplitudes are optimal to achieve fine control, a single output source switched across electrodes may also be used, although with less fine control in programming. Mixed current and voltage regulated devices may also be used with the invention. Further details discussing the detailed structure and function of IPGs are described more fully in U.S. Pat. Nos. 6,516,227 and 6,993,384, which are expressly incorporated herein by reference.

It should be noted that rather than an IPG, the SCS system 10 may alternatively utilize an implantable receiver-stimulator (not shown) connected to the neurostimulation leads 12. In this case, the power source, e.g., a battery, for powering the implanted receiver, as well as control circuitry to command the receiver-stimulator, will be contained in an external controller inductively coupled to the receiver-stimulator via an electromagnetic link. Data/power signals are transcutaneously coupled from a cable-connected transmission coil placed over the implanted receiver-stimulator. The implanted receiver-stimulator receives the signal and generates the stimulation in accordance with the control signals.

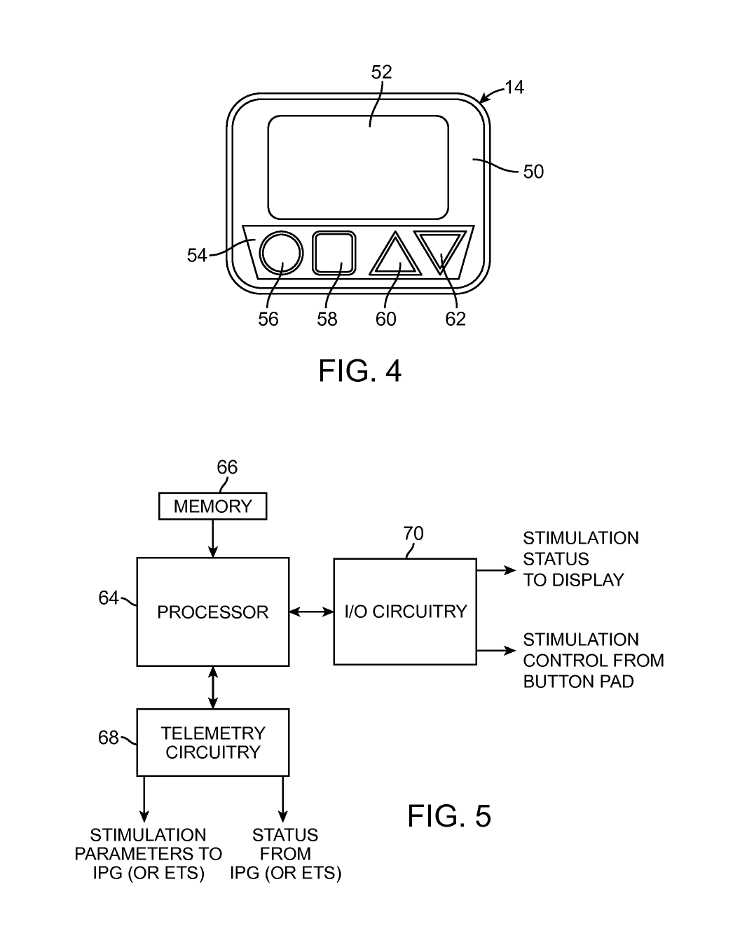

Referring now to FIG. 4, one exemplary embodiment of an RC 16 will now be described. As previously discussed, the RC 16 is capable of communicating with the IPG 14, CP 18, or ETS 20. The RC 16 comprises a casing 50, which houses internal componentry (including a printed circuit board (PCB)), and a lighted display screen 52 and button pad 54 carried by the exterior of the casing 50. In the illustrated embodiment, the display screen 52 is a lighted flat panel display screen, and the button pad 54 comprises a membrane switch with metal domes positioned over a flex circuit, and a keypad connector connected directly to a PCB. In an optional embodiment, the display screen 52 has touch screen capabilities. The button pad 54 includes a multitude of buttons 56, 58, 60, and 62, which allow the IPG 14 to be turned ON and OFF, provide for the adjustment or setting of stimulation parameters within the IPG 14, and provide for selection between screens.

In the illustrated embodiment, the button 56 serves as an ON/OFF button that can be actuated to turn the IPG 14 ON and OFF. The button 58 serves as a select button that allows the RC 16 to switch between screen displays and/or parameters. The buttons 60 and 62 serve as up/down buttons that can be actuated to increment or decrement any of stimulation parameters of the pulse generated by the IPG 14, including pulse amplitude, pulse width, and pulse rate. For example, the selection button 58 can be actuated to place the RC 16 in a “Pulse Amplitude Adjustment Mode,” during which the pulse amplitude can be adjusted via the up/down buttons 60, 62, a “Pulse Width Adjustment Mode,” during which the pulse width can be adjusted via the up/down buttons 60, 62, and a “Pulse Rate Adjustment Mode,” during which the pulse rate can be adjusted via the up/down buttons 60, 62. Alternatively, dedicated up/down buttons can be provided for each stimulation parameter. Rather than using up/down buttons, any other type of actuator, such as a dial, slider bar, or keypad, can be used to increment or decrement the stimulation parameters. Further details of the functionality and internal componentry of the RC 16 are disclosed in U.S. Pat. No. 6,895,280, which has previously been incorporated herein by reference.

Referring to FIG. 5, the internal components of an exemplary RC 16 will now be described. The RC 16 generally includes a processor 64 (e.g., a microcontroller), memory 66 that stores an operating program for execution by the processor 64, as well as stimulation parameter sets in a navigation table (described below), input/output circuitry, and in particular, telemetry circuitry 68 for outputting stimulation parameters to the IPG 14 and receiving status information from the IPG 14, and input/output circuitry 70 for receiving stimulation control signals from the button pad 54 and transmitting status information to the display screen 52 (shown in FIG. 4). As well as controlling other functions of the RC 16, which will not be described herein for purposes of brevity, the processor 64 generates new stimulation parameter sets in response to the user operation of the button pad 54. These new stimulation parameter sets would then be transmitted to the IPG 14 (or ETS 20) via the telemetry circuitry 68. Further details of the functionality and internal componentry of the RC 16 are disclosed in U.S. Pat. No. 6,895,280, which has previously been incorporated herein by reference.

As briefly discussed above, the CP 18 greatly simplifies the programming of multiple electrode combinations, allowing the user (e.g., the physician or clinician) to readily determine the desired stimulation parameters to be programmed into the IPG 14, as well as the RC 16. Thus, modification of the stimulation parameters in the programmable memory of the IPG 14 after implantation is performed by a user using the CP 18, which can directly communicate with the IPG 14 or indirectly communicate with the IPG 14 via the RC 16. That is, the CP 18 can be used by the user to modify operating parameters of the electrode array 26 near the spinal cord.

As shown in FIG. 2, the overall appearance of the CP 18 is that of a laptop personal computer (PC), and in fact, may be implemented using a PC that has been appropriately configured to include a directional-programming device and programmed to perform the functions described herein. Thus, the programming methodologies can be performed by executing software instructions contained within the CP 18. Alternatively, such programming methodologies can be performed using firmware or hardware. In any event, the CP 18 may actively control the characteristics of the electrical stimulation generated by the IPG 14 (or ETS 20) to allow the optimum stimulation parameters to be determined based on patient feedback and for subsequently programming the IPG 14 (or ETS 20) with the optimum stimulation parameters.

To allow the user to perform these functions, the CP 18 includes a mouse 72, a keyboard 74, and a programming display screen 76 housed in a case 78. It is to be understood that in addition to, or in lieu of, the mouse 72, other directional programming devices may be used, such as a trackball, touchpad, joystick, or directional keys included as part of the keys associated with the keyboard 74. The CP 18 further includes detection circuitry 80 capable of detecting an actuation event on the display screen 76. Such actuation event may include placing at least one pointing element (not shown) in proximity to at least one graphical object displayed on the display screen 76, as well as possibly other events involving the point element(s), such as moving the pointing element(s) across the screen or clicking or tapping with the pointing element(s), as will be described in further detail below.

In the preferred embodiments described below, the display screen 76 takes the form of a digitizer touch screen, which may either passive or active. If passive, the display screen 76 includes detection circuitry that recognizes pressure or a change in an electrical current when a passive device, such as a finger or non-electronic stylus, contacts the screen. If active, the display screen 76 includes detection circuitry that recognizes a signal transmitted by an electronic pen or stylus. In either case, the detection circuitry 80 is capable of detecting when a physical pointing device (e.g., a finger, a non-electronic stylus, or an electronic stylus) is in close proximity to the screen, whether it be making physical contact between the pointing device and the screen or bringing the pointing device in proximity to the screen within a predetermined distance, as well as detecting the location of the screen in which the physical pointing device is in close proximity. When the pointing device touches or otherwise is in close proximity to the screen, the graphical object on the screen adjacent to the touch point is “locked” for manipulation, and when the pointing device is moved away from the screen the previously locked object is unlocked.

In some embodiments described below, the display screen 76 takes the form of a conventional screen, in which case, the pointing element is not an actual pointing device like a finger or stylus, but rather is a virtual pointing device, such as a cursor controlled by a mouse, joy stick, trackball, etc.

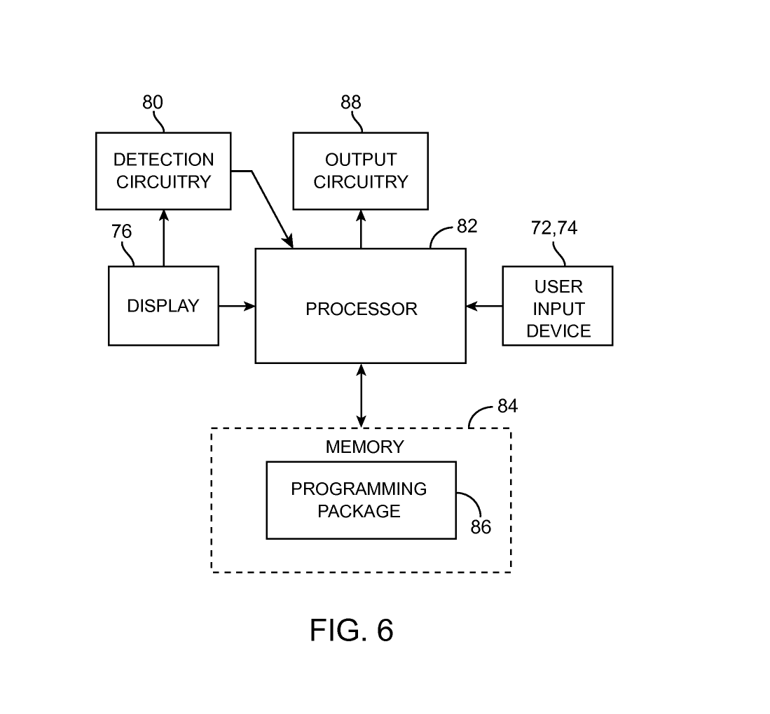

As shown in FIG. 6, the CP 18 generally includes a processor 82 (e.g., a central processor unit (CPU)) and memory 84 that stores a stimulation programming package 86, which can be executed by the processor 82 to allow the user to program the IPG 14, and RC 16. The CP 18 further includes output circuitry 88 (e.g., via the telemetry circuitry of the RC 16) for downloading stimulation parameters to the IPG 14 and RC 16 and for uploading stimulation parameters already stored in the memory 66 of the RC 16, via the telemetry circuitry 68 of the RC 16.

Execution of the programming package 86 by the processor 82 provides a multitude of display screens (not shown) that can be navigated through via use of the mouse 72. These display screens allow the clinician to, among other functions, to select or enter patient profile information (e.g., name, birth date, patient identification, physician, diagnosis, and address), enter procedure information (e.g., programming/follow-up, implant trial system, implant IPG, implant IPG and lead(s), replace IPG, replace IPG and leads, replace or revise leads, explant, etc.), generate a pain map of the patient, define the configuration and orientation of the leads, initiate and control the electrical stimulation energy output by the leads 12, and select and program the IPG 14 with stimulation parameters in both a surgical setting and a clinical setting. Further details discussing the above-described CP functions are disclosed in U.S. patent application Ser. No. 12/501,282, entitled “System and Method for Converting Tissue Stimulation Programs in a Format Usable by an Electrical Current Steering Navigator,” and U.S. patent application Ser. No. 12/614,942, entitled “System and Method for Determining Appropriate Steering Tables for Distributing Stimulation Energy Among Multiple Neurostimulation Electrodes,” which are expressly incorporated herein by reference.

Most pertinent to the present inventions, execution of the programming package 86 provides a more intuitive user interface that conveniently allows a user to program the IPG 14 (or ETS 20) in a manner that emulates an abstraction of a stimulation target (e.g., an ideal multipole, such as a bipole or tripole, an ideal electrical field, or an ideal stimulation region).

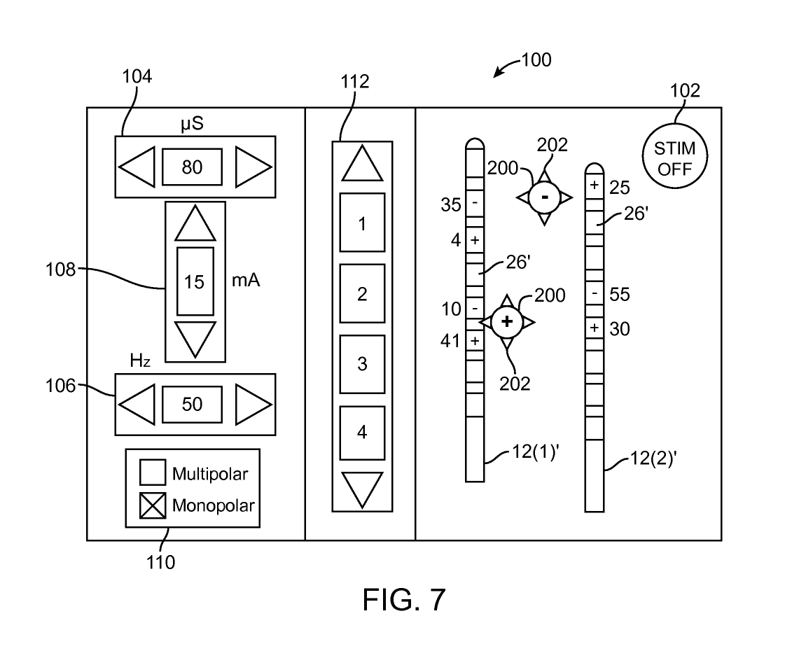

Referring now to FIG. 7, an exemplary programming screen 100 generated by the CP 16 to allow a user to program the IPG 14 (or ETS 20) will now be described. Because the display screen 76 is a digitizer touchscreen, the various graphical control elements described herein can be actuated by touching the respective control elements on the screen or otherwise bringing a physical pointing device into proximity with the respective control elements.

The programming screen 100 comprises a stimulation on/off control 102 that can be alternately actuated initiate or cease the delivery of electrical stimulation energy from the IPG 14. The programming screen 100 further includes various stimulation parameter controls that can be operated by the user to manually adjust stimulation parameters. In particular, the programming screen 100 includes a pulse width adjustment control 104 (expressed in microseconds (μs)), a pulse rate adjustment control 106 (expressed in Hertz (Hz)), and a pulse amplitude adjustment control 108 (expressed in milliamperes (mA)). Each control includes a first arrow that can be actuated to decrease the value of the respective stimulation parameter and a second arrow that can be actuated to increase the value of the respective stimulation parameter. The programming screen 100 also includes multipolar/monopolar stimulation selection control 110, which includes check boxes that can be alternately actuated by the user to selectively provide multipolar or monopolar stimulation. The programming screen 100 also includes an electrode combination control 112 having arrows that can be actuated by the user to select one of three different electrode combinations 1-4. Each of the electrode combinations 1-4 can be created using a variety of control elements.

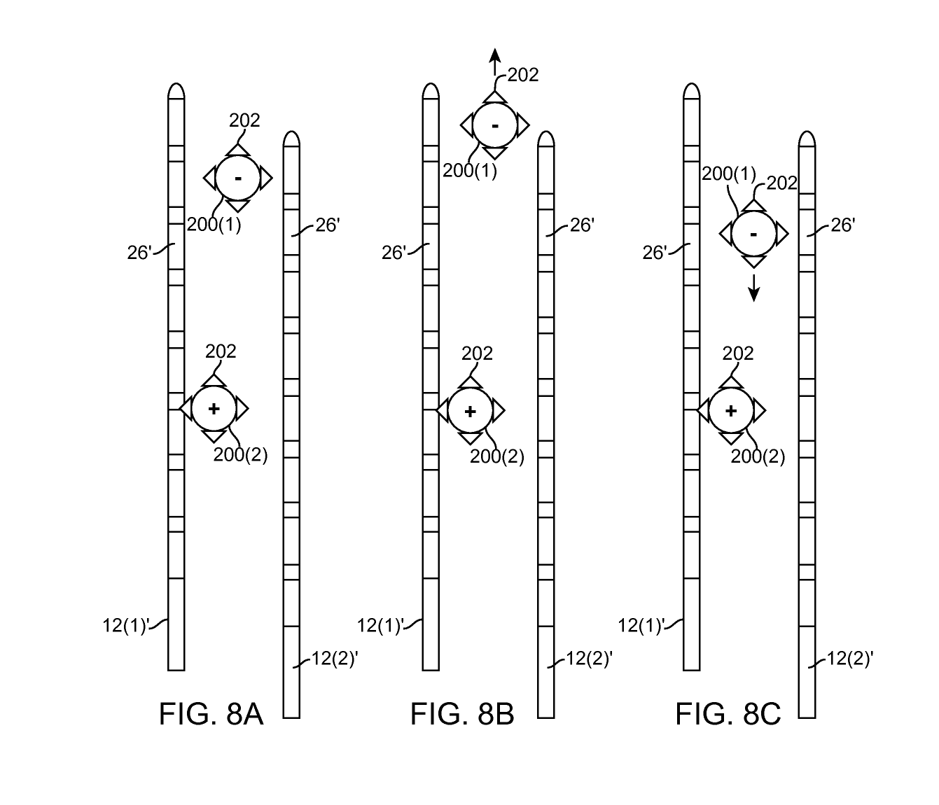

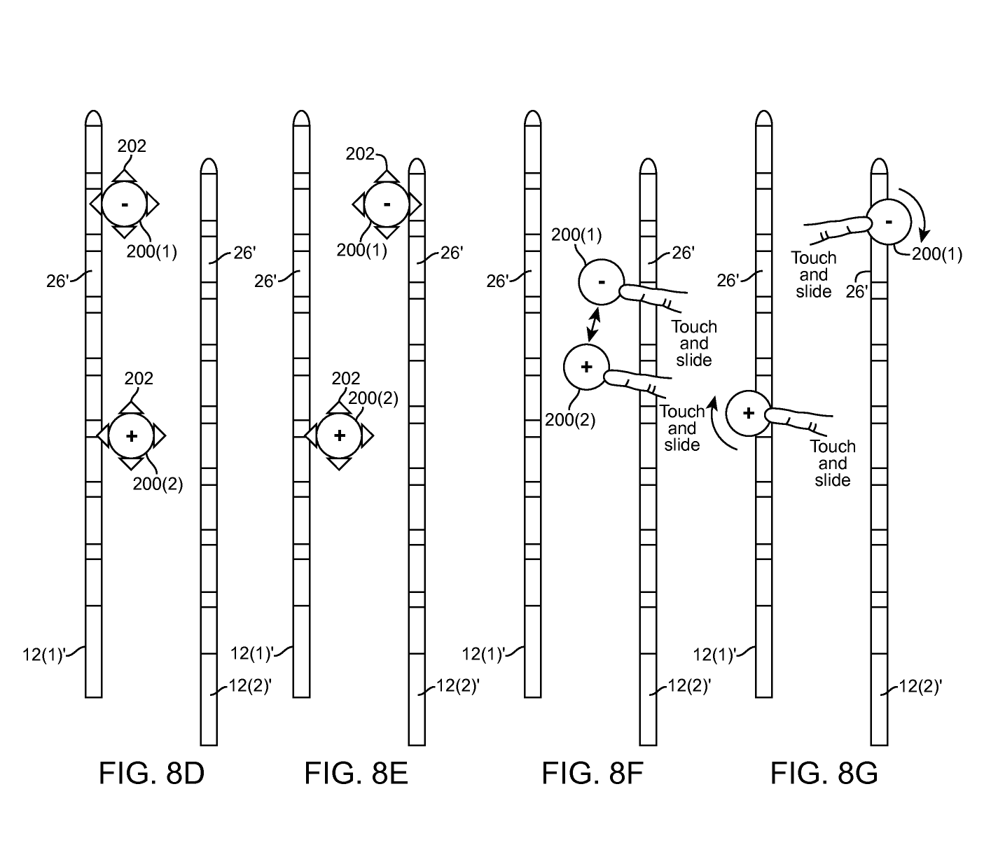

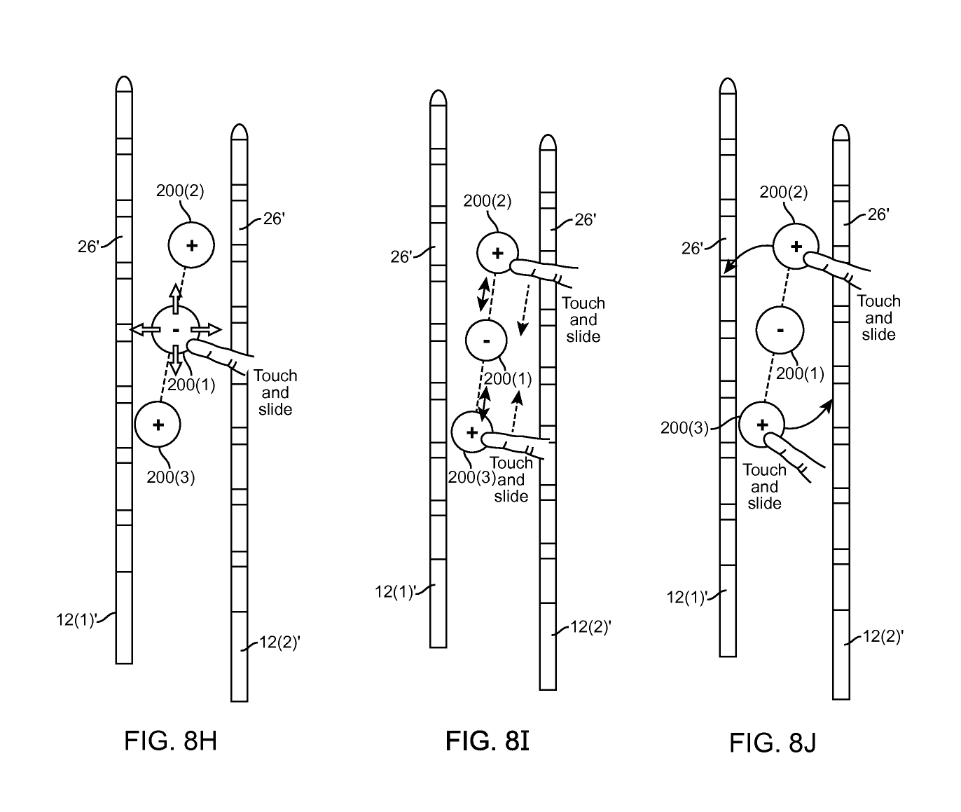

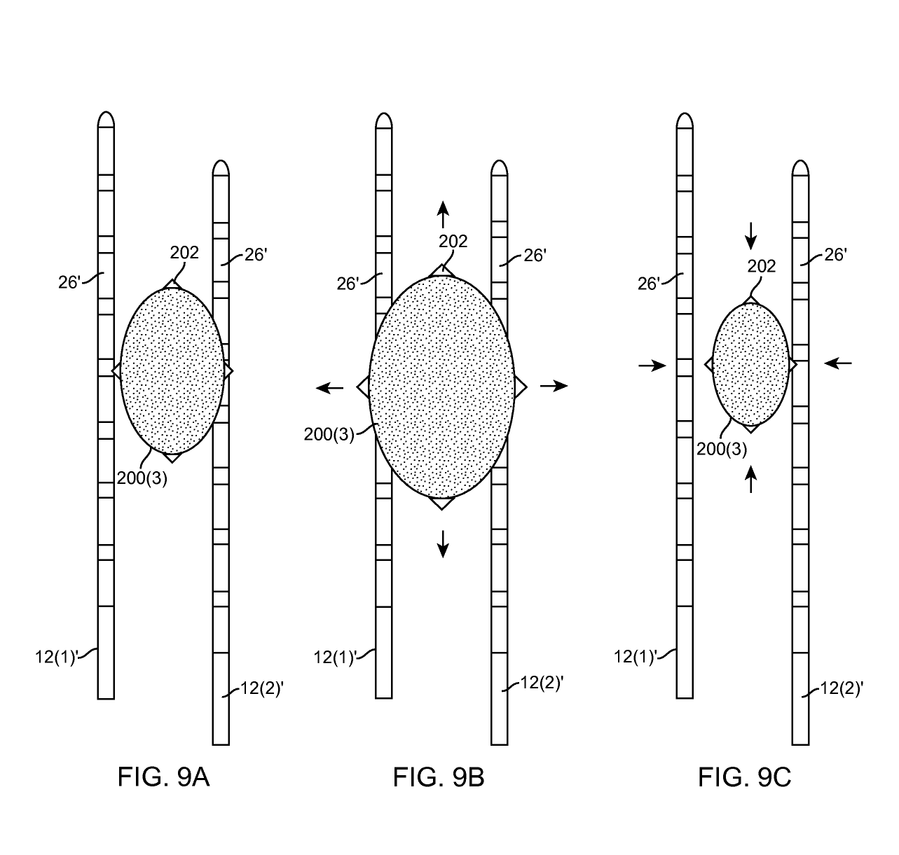

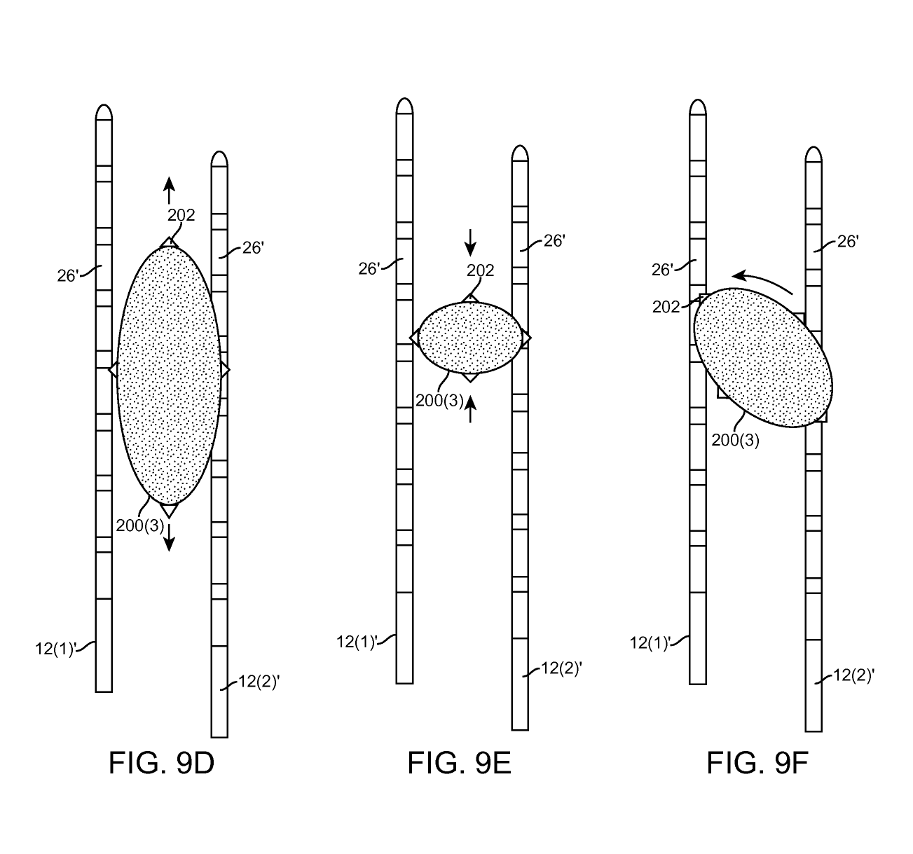

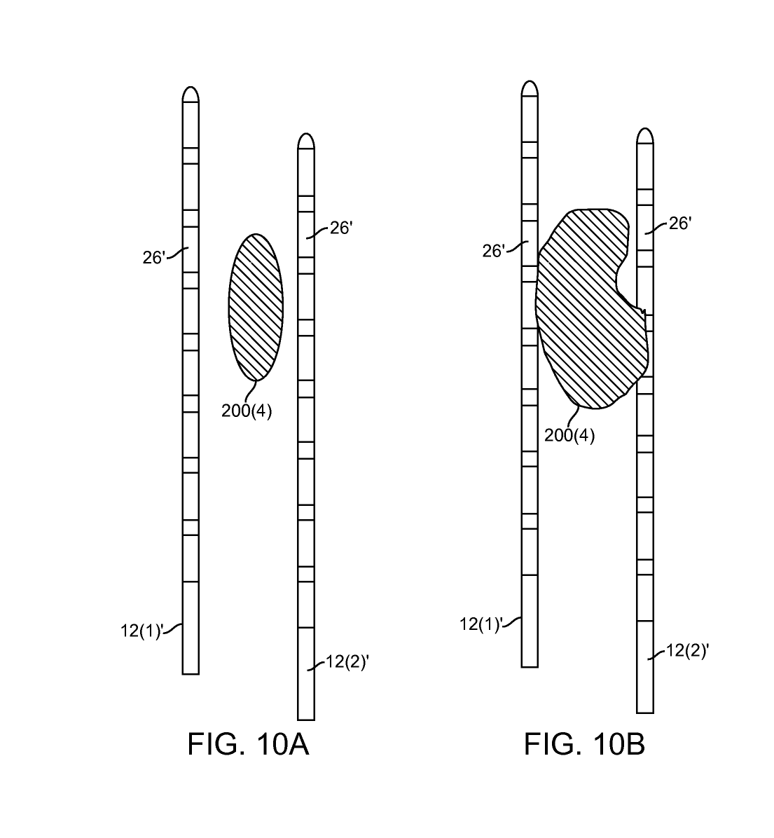

The programming screen 100 displays a two-dimensional graphical rendering of the leads 12′ including electrodes 26′. As shown, the polarities and associated fractionalized current values are displayed next to the activated electrodes 26′. The programming screen 100 further displays at least one target object 200 representing an abstraction of a stimulation target, which for the purposes of this specification, can be defined as any abstract information at a level higher than the display of conventional stimulation parameters (pulse amplitude, pulse width, pulse rate, electrode combinations, etc.) that provides the user with a visualization of a desired tissue region relative to the leads 12 to be stimulated. In alternative embodiments, the graphical rendering of the leads 12 may be three-dimensional. The programming screen 100 may optionally comprise a graphical anatomical region (not shown) displayed relative to the graphical leads 12′. As briefly discussed above, a stimulation target abstraction can be an ideal multi-pole (FIGS. 8A-8J), in which case there will be multiple target objects (in the illustrated embodiment, objects 200(1) and 200(2) representing an ideal bipole or objects 200(1), 200(2), and 200(3) representing an ideal tripole), or an ideal electrical field (FIGS. 9A-9E) or ideal stimulation region (FIG. 10A-10B), in which case there may be a single target object or multiple target objects (in the illustrated embodiment, a single object 200(3) representing an ideal electrical field or a single object 200(4) representing an ideal stimulation region), depending on the number of targeted tissue regions desired to be stimulated.

The processor 82 is configured for manipulating the target object(s) 200 in response to the detection of the actuation event by the detection circuitry 80 briefly discussed above, thereby modifying the stimulation target abstraction. The processor 82 is further configured for generating a set of stimulation parameters that emulates the modified stimulation target abstraction; for example by computationally determining the fractionalized current values on each of the electrodes in a manner that emulates the ideal bipole or tripole, as described in U.S. Provisional Application Ser. No. 61/257,753, which was previously incorporated herein by reference. In the case where each target object 200 represents an ideal electrical field, the technique described in U.S. Provisional Patent Application Ser. No. 61/257,753 models an ideal electrical field from the ideal bipole or tripole as an intermediate solution, and therefore, may compute the fractionalized current values on the electrodes to emulate the ideal electrical field. In the case where each target object 200 represents an ideal stimulation region, an ideal electrical field may be easily derived from the ideal stimulation region, and the fractionalized current values may be computed to emulate the ideal electrical field.

The programming screen 100 also displays a plurality of graphical control elements 202 in the form of arrows surrounding each of the objects 200 representing the ideal bipole. In this case, there are four arrows: an up arrow, a bottom arrow, a left arrow, and a right arrow. A pointing element (not shown) may be placed on any of the control elements 202 to perform the actuation event. In the case of a digitizer touch screen, the pointing element will be an actual pointing element (e.g., a finger or active or passive stylus) that can be used to physically tap the screen above the respective control element 202. In the case of a conventional screen, the pointing element will be a virtual pointing element (e.g., a cursor) that can be used to graphically click on the respective control element 202.

In either case, in response to the actuation event, the processor 82 will manipulate the object 200 relative to the leads 12′. The processor 82 will also displace all of the control elements 202 associated with the respective object 200 in unison with the manipulation of the object 200, such that the control elements 202 remain displaced adjacent the respective object 200.

In one advantageous embodiment, the processor 82 generates the stimulation parameter set in accordance with a first current steering algorithm (e.g., a weaving current steering algorithm) in response to the detection of the actuation event (in this case, the actuation of one of the control elements 202). The user interface of the CP 18 can also have another set of control elements (e.g., arrow keys on the keyboard or graphical arrows located remotely from the objects 200) that can be actuated, and the processor 82 may be configured for generating another stimulation parameter set in accordance with a second current steering algorithm (e.g., a panning current steering algorithm) in response to the actuation of one of the other control elements.

Having generally described the operation of the processor 82 in the manipulation of the graphical objects 200 in response to actuation events, specific examples of the manner in which the processor 82 manipulates the graphical objects 200 in response to various types of actuation events will now be described.

With reference to FIGS. 8A-8E, the displacement of an object 200(1) representing one of the poles of an ideal bipole in response to the actuation of one of the control arrows 202 will be described. In response to the actuation of one of the control arrows 202, the processor 82 will displace the object 200 relative to the leads 12′ in the direction of the arrow 202 that is actuated. For example, assuming the initial object 200(1) position illustrated in FIG. 8A, the top control arrow 202 associated with the object 200(1) may be actuated to displace the object 200(1) upward relative to the leads 12′, as shown in FIG. 8B; the bottom control arrow 202 associated with the object 200(1) may be actuated to displace the object 200(1) downward relative to the leads 12′, as shown in FIG. 8C; the left control arrow 202 associated with the object 200(1) may be actuated to displace the object 200(1) to the left relative to the leads 12′, as shown in FIG. 8D; and the right control arrow 202 associated with the object 200(1) may be actuated to displace the object 200(1) to the right relative to the leads 12′, as shown in FIG. 8E. Of course, any of the arrows 202 associated with the object 200(2) representing the positive pole may be actuated to displace that object 200 in the direction of the actuated arrow.

In an alternative embodiment, visual graphical control elements separate from the objects are not utilized. Instead, the pointing element (whether actual or virtual) is simply placed over the object 200 and displaced across the screen to display the object 200. In the case where the display screen 76 is a digitizer touch screen, the actuating event may alternatively include concurrently placing two actual pointing elements (e.g., fingers or styluses) respectively on a plurality of objects 200 and moving the actual pointing elements relative to each other. The pointing elements can be concurrently placed on the respective objects 200 by, e.g., placing one pointing element on one object 200 and then placing another pointing element on the other object 200 while leaving the first pointing element on the first object 200, or by simply simultaneously placing the pointing elements respectively on the objects 200.

For example, the respective objects 200 can be linearly moved away from each other by linearly moving the actual pointing elements (shown as fingers) away from each other or can be linearly moved toward each other by moving the actual pointing elements toward each other. For example, two actual pointing elements can be respectively placed on the objects 200(1) and 200(2) and moved toward each other to move the objects 200(1) and 200(2) toward each other, as shown in FIG. 8F. As another example, the respective objects 200 can be displaced relative to each other by rotating the objects 200 about a point between the objects by rotating the actual pointing elements about the point. For example, two actual pointing elements can be respectively placed on the objects 200(1) and 200(2) and rotated about a point to rotate the objects 200(1) and 200(2) about the point, as shown in FIG. 8G.

Referring to FIGS. 8H-8J, in still another embodiment, a tripole consisting of a middle cathode (object 200(1) and two flanking anodes (objects 200(2) and 200(3)) can be manipulated using actual pointing elements (shown as fingers) in accordance with a general rule that the entire tripole will be moved with the movement of the cathode, and the anodes can be moved independently of each other relative to the cathode or dependently symmetrically opposite with respect to the cathode. Thus, all the objects 200(1)-200(3) will be moved in unison when an actual pointing element is placed on the object 200(2) and moved, but only the object 200(1) or 200(3) will be moved when an actual pointing is placed over this object and moved.

For example, a single actual pointing element can be placed on the object 200(1) and moved to move the objects 200(1)-200(3) in unison with the movement of the object 200(1), as shown in FIG. 8H. An actual pointing element can be placed on the objects 200(2) and/or an actual pointing element can be placed on the object 200(3) and independently (or dependently symmetric) moved toward or away from the object 200(1), as shown in FIG. 8I. Two actual pointing elements can be respectively placed on the objects 200(1) and 200(2) and rotated about the object 200(3) to rotate the objects 200(1) and 200(2) about object 200(3), as shown in FIG. 8J.

In the embodiment illustrated in FIGS. 9A-9E, the programming screen 100 also displays a plurality of graphical control elements 202 in the form of arrows surrounding the object 200(3) representing the electrical field. The object 200(3) may be displaced relative to the leads 12′ in the same manner described above with respect to the object 200(1).

In addition, if the display screen 76 is a digitizer touch screen, the actuating event may alternatively include concurrently placing two actual pointing elements (e.g., fingers or styluses) respectively on oppositely disposed control elements 202 and moving the actual pointing elements relative to each other to expand the respective object 200(3) (moving the actual pointing elements away from each other) or contract the respective object 200(3) (moving the actual pointing elements toward each other). For example, the actual pointing elements can be placed on the up and down arrows 202 and moved away from each other to expand the object 200(3) (FIG. 9B) or moved toward each other to contract the object 200(3) (FIG. 9C). The pointing elements can be concurrently placed on the respective control elements 202 by, e.g., placing one pointing element on one control element 202 and then placing another pointing element on the other control element 202 while leaving the first pointing element on the first control element 202, or by simply simultaneously placing the pointing elements respectively on the control elements 202.

Thus, it can be appreciated that the control elements 202 can be used for selectively displacing the object 200(3) (e.g., by tapping or clicking the control elements 202 with a pointing element) or shaping the geometry of the object 200(3), and in this case expanding or contracting the object 200(3), by placing two pointing elements on the control elements 202 and displacing the control elements 202 relative to each other.

In one embodiment, displacement of the two actual pointing elements relative to each other will equally expand or contract in all directions, as shown in FIGS. 9B and 9C. For example, placing the actual pointing elements respectively on the up and down arrows 202 and moving them away from each other will expand the object 200(3) in both the vertical and horizontal directions.

In another embodiment, the object 200(3) can be expanded or contracted in only one direction by placing and holding another two pointing elements on the oppositely disposed arrows 202 corresponding to the direction that the user does not want to change, while displacing the first two pointing elements relative to each other. For example, two pointing elements can be respectively placed on the up and down arrows 202, and two pointing elements can be respectively placed on the left and right down arrows 202. Then the two pointing elements on the up and down arrows 202 can be displaced relative to each other, while the two pointing elements on the left and right arrows 202 can be maintained. As a result, the object 200(3) may be expanded (FIG. 9D) or contracted (FIG. 9E) only in the vertical direction. Alternatively, the object 202 will only be expanded or contracted in the direction along the pair of oppositely disposed arrows that are actuated by the pointing elements without regard to whether the pointing elements are placed on the other pair of oppositely disposed arrows 202.

In still another embodiment, the object 200 can be shaped by placing a pointing element on the margin of the object 200 and moving the pointing element to displace the margin. Any segment of the margin of the object 200 can be displaced in this manner, so that the object 200 can be shaped into any form desired by the user. For example, as shown in FIG. 10B, a pointing element can be placed on various regions of the margin of the object 200(4) representing the ideal stimulation region and displaced to amorphously form the object 200(4).

Although the foregoing techniques have been described as being implemented in the CP 18, it should be noted that this technique may be alternatively or additionally implemented in the RC 16.

Although particular embodiments of the present inventions have been shown and described, it will be understood that it is not intended to limit the present inventions to the preferred embodiments, and it will be obvious to those skilled in the art that various changes and modifications may be made without departing from the spirit and scope of the present inventions. Thus, the present inventions are intended to cover alternatives, modifications, and equivalents, which may be included within the spirit and scope of the present inventions as defined by the claims.

What is claimed is:

1. An external control device for use with a neurostimulation device having a plurality of electrodes carried by at least one neurostimulation lead, comprising:
a display screen configured for displaying at least one object relative to a graphical representation of the at least one neurostimulation lead, the at least one object representing an abstraction of a stimulation target;
detection circuitry configured for detecting an actuation event that includes placing at least one pointing element in proximity to the at least one object;
processing circuitry configured for manipulating the at least one object on the display screen in response to the detection of the actuation event, thereby modifying the stimulation target abstraction, and for generating a set of stimulation parameters that emulates the modified stimulation target abstraction; and
output circuitry configured for transmitting the set of stimulation parameters to the neurostimulation device.

2. The external control device of claim 1, wherein the actuation event further includes moving the at least one pointing element across the display screen.

3. The external control device of claim 1, wherein the display screen is configured for displaying a graphical control element adjacent each of the at least one object, the actuation event includes placing the at least one pointing element respectively on the at least one control element, and the processing circuitry is configured for displacing the at least one graphical control element in unison with the manipulation of the at least one object, such that the graphical control element remains displayed adjacent the at least one manipulated object.

4. The external control device of claim 3, wherein the actuation event includes clicking or tapping the at least one control element.

5. The external control device of claim 3, further comprising another control element configured for being actuated, wherein the processing circuitry is configured for generating the stimulation parameter set in accordance with a first current steering algorithm in response to the detection of the actuation event, and for generating another stimulation parameter set in accordance with a second current steering algorithm in response to the actuation of the other control element.

6. The external control device of claim 1, wherein each of the at least one pointing element is a virtual pointing element, and the actuation event includes graphically placing the at least one virtual pointing element in proximity to the at least one object.

7. The external control device of claim 1, wherein each of the at least one pointing element is an actual pointing element, and the actuation event includes physically placing the at least one actual pointing element in proximity to the at least one object.

8. The external control device of claim 7, wherein the at least one actual pointing element comprises a plurality of actual pointing elements, the actuating event includes placing the actual pointing elements concurrently in proximity to the at least one object and moving the actual pointing elements relative to each other across the display screen.

9. The external control device of claim 8, wherein the at least one object comprises a plurality of objects, the actuating event includes placing the actual pointing elements concurrently in proximity to the respective objects, and the processing circuitry is configured for manipulating the objects on the display screen by moving the objects relative to each other.

10. The external control device of claim 8, wherein the at least one object comprises a single object, the actuating event includes placing the actual pointing elements concurrently in proximity to the single object, and the processing circuitry is configured for manipulating the object on the display screen by modifying the geometry of the object.

11. The external control device of claim 1, wherein the processing circuitry is configured for manipulating the at least one object on the display screen by displacing the at least one object on the display screen.

12. The external control device of claim 11, wherein the at least one object comprises a plurality of objects, and the processing circuitry is configured for displacing the objects relative to each other.

13. The external control device of claim 1, wherein the processing circuitry is configured for manipulating the at least one object on the display screen by modifying the geometry of the at least one object.

14. The external control device of claim 13, wherein the processing circuitry is configured for modifying the shape of the at least one object on the display screen by expanding or contracting the at least one object.

15. The external control device of claim 1, wherein the stimulation target abstraction is an ideal multi-pole.

16. The external control device of claim 1, wherein the stimulation target abstraction is an ideal electrical field.

17. The external control device of claim 1, wherein the stimulation target abstraction is an ideal stimulation region.

18. The external control device of claim 1, wherein the output circuitry comprises telemetry circuitry.

19. The external control device of claim 1, further comprising a housing containing the display screen, control element, processing circuitry, and output circuitry.
